(19) United States
(12) Patent Application Publication
(10) Pub. No.: US 2013/0102645 A1
(43) Pub. Date: Apr. 25, 2013

(54) Methods and compositions for controlling vascularization in ophthalmological and dermatological diseases

(71) Applicant: Burkhard Jansen, Vienna (AT)

(72) Inventor: Burkhard Jansen, Vienna (AT)

(73) Assignee: AVIENNE PHARMACEUTICALS GMBH, Vienna (AT)

(21) Appl. No.: 13/506,654

(22) Filed: May 7, 2012

Related U.S. Application Data
(60) Provisional application No. 61/549,517, filed on Oct. 20, 2011.

(51) Int. Cl. A61K 31/426; A61P 17/00; A61P 27/02

(52) U.S. Cl. 514/369

(57) ABSTRACT

A method for controlling vascularization in a patient's eye or skin includes administering to the patient's eye or skin a pharmaceutical composition having

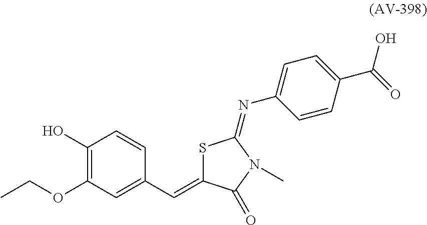 [SMILES: CCOc1cc(/C=C2\S/C(=N/c3ccc(C(=O)O)cc3)N(C)C2=O)ccc1O]

or a pharmaceutically acceptable salt, hydrate, enantiomer, diastereomer, racemate or mixtures of stereoisomers thereof, wherein the patient has a disease or disorder associated with vascularization in the eye or skin or wherein said patient is at risk for developing a disease or disorder associated with vascularization of the eye or skin.

### CROSS-REFERENCE TO RELATED APPLICATIONS

This application claims priority to U.S. Provisional Application No. 61/549,617 filed Oct. 20, 2011, the entire contents of which is specifically incorporated herein by reference without disclaimer.

### BACKGROUND OF THE INVENTION

1. Field of the Invention

The present invention is directed to methods and compositions for controlling vascularization in ophthalmological and dermatological diseases and disorders.

2. Description of Related Art

Endothelial cells are oblong shaped cells that line the lumen of all blood vessels as a single squamous epithelial cell layer. These cells play a major role in vascular biology under normal or pathological conditions, including the control of blood pressure (vasoconstriction and vasodilatation), blood clotting (thrombosis, fibrinolysis), and formation of new blood vessels. In small capillaries endothelial cells may be the only cell type present. New vessel formation or neo-angiogenesis plays an important role in a variety of disease states of the eye and skin. For all types of angiogenesis, that is the formation of vascular structures and vessel growth, endothelial cells are essential. Inhibition of adhesion, growth and survival of endothelial cells by blocking integrin function therefore is a rational strategy to inhibit diseases which depend on angiogenesis.

The basic anatomic structures of the eye may generally be divided into an anterior segment and a posterior segment. The anterior segment is the front third of the eye that includes the structures in front of the vitreous humor that includes the cornea, iris, ciliary body and lens. The posterior segment is the back two-thirds of the eye that includes the anterior hyaloid membrane and all of the optical structures behind it, including the vitreous humor, retina, choroid, and optic nerve. New vessel formation or neo-angiogenesis has been implicated in a variety of disease states in both the anterior and posterior segment.

For instance, regarding the anterior segment, several corneal diseases can lead to pathological corneal neovascularization. Neovascularization differs from angiogenesis in that angiogenesis is mainly characterized by the protrusion and outgrowth of capillary buds and sprouts from pre-existing blood vessels.

These can be grouped into diseases causing corneal inflammation (e.g., bacterial and viral forms of keratitis), diseases interfering with the limbal barrier between normally avascular cornea and physiologically vascularized conjunctiva (e.g., chemical burns or inherited forms of limbal deficiency) and finally diseases presumably leading to corneal hypoxia (i.e., contact lenses with low oxygen penetration). Corneal neovascularization is associated with the second most common cause of blindness worldwide (trachoma) and also with the most common form of corneal blindness in industrialized countries, such as herpetic keratitis. In fact, corneal neovascularization does not only seem to be a sequel of certain inflammatory corneal diseases, but also may be causative for autoimmune forms of herpetic keratitis (see also Regenfuss B et al, 2008). Corneal neovascularization is also often the result of inflammation, chemical burns, and conditions related to hypoxia. These conditions are currently treated by indirect angiogenesis inhibitors such as steroids and immunosuppressants.

Diseases associated with neovascularization of the posterior segment are often treated with anti-VEGF inhibitors. VEGF inhibitors such as Bevacizumab (Avastin®), Ranibizumab (Lucentis®), and Pegaptanib (Macugen®) are now a mainstay for treating neovascular forms of age-related maculopathy. They are also used for treating diabetic retinopathy, retinal venous occlusions, and neovascular glaucoma. The growth of abnormal, leaky blood vessels is a cause of several eye diseases including AMD, proliferative diabetic retinopathy (PDR), and retinopathy of prematurity (ROP). However, the VEGF inhibitors commonly used to treat these diseases are generally complex biologics based on monoclonal antibody or oligonucleotide aptamer technology. And treatment with VEGF inhibitors may only work for a limited period of time. Therefore antiangiogenic strategies based on small molecule pharmaceuticals could be particularly beneficial in preventing and treating the progression of these diseases.

A number of dermatological diseases are associated with pathologically increased blood vessel formation. Although angiogenesis occurs in the skin during physiological processes, for example in the anagen stage of the hair cycle, a sustained and significant increase in new blood vessels in the skin is seen predominately in cutaneous diseases. Indeed, prominent blood vessels are a clinical characteristic of diseases such as rosacea, psoriasis and in skin tumors such as basal cell carcinoma. Other dermatological diseases associated with new blood vessel formation include dermatitis (including atopic dermatitis and eczematous dermatitis), autoimmune skin diseases, acute and chronic urticaria, scleroderma, vasculitis, port-wine stains, blue rubber bleb syndrome, Osler-Weber-Rendu syndrome, Sturge-Weber syndrome Klippel-Trenaunay syndrome, non-melanoma skin cancer other than basal cell carcinoma, malignant melanoma, haemangiomas, angiosarcoma, pyogenic granuloma, viral warts, and keloid scars.

A variety of functions of endothelial cells are regulated by a class of proteins expressed on the surface of endothelial cells, which are termed integrins. Integrins belong to a family of membrane-spanning adhesion receptors. Integrins mediate intracellular signalling events controlling cell migration, proliferation, metastasis and survival in endothelial as well as in other cell types (Aplin A E et al (1998), Howe A et al (1998), Schwartz M A et al (2000), Stromblad S et al (1996), Zedith J E et al (1993)). Some integrins have been demonstrated to play an important role in angiogenesis by interacting with a number of extracellular matrix proteins, such as vitronectin, fibrinogen, fibronectin, thrombin, thrombospondin, and other factors (Cheresh D A et al (1987), Ruoslahti E (1996)). Some integrins are able to interact with protein domains containing the Arg-Gly-Asp (RGD) amino acid sequence characteristic for various extracellular matrix-associated adhesive glycoproteins (Cheresh D A et al (1987), Ruoslahti E (1996)).

Integrins are composed of heterodimers of noncovalently linked alpha and beta subunits (Hynes R O (2003), and references therein). Combinations of these subunits are able form a variety of heterodimeric receptors with different ligand binding properties as well as diverse physiological functions. To date, at least 18 different α- and eight β-subunits have been identified. One such integrin, αvβ3, is also known as the vitronectin receptor and consists of a 125 kDa αv subunit and a 105 kDa β3 subunit. This receptor has been implicated in several pathophysiological processes such as rheumatoid arthritis and in other diseases associated with neovascularization, inflammation and/or increased osteoclast activity.

While the expression of certain types of integrins, such as αvβ3, is low on normal epithelial cells and non-dividing endothelial cells, it is up-regulated on activated endothelial cells in the vasculature of tumors and expressed on tumor cells themselves. Several treatment strategies based on integrin inhibition (antibodies, small molecules, synthetic peptides) are now being investigated in the treatment of cancer. Several anti-angiogenic compounds based on integrin inhibition are in clinical studies such as Abegrin™ (MEDI-522, formerly Vitaxin) or cilengitide (EMD 121974, cycloL-Arg-Gly-L-Asp-D-Phe-N [Me] L-Val; Merck KGaA) (Cai W et al (2006), Eskens F A et al (2003), Mulgrev K et al (2006), Nabors L B et al (2007), Smith J W (2003)). Cilengitide, the most advanced αvβ3 antagonist currently in phase III clinical trials, is capable of inducing apoptosis in brain tumor cells (Taga T et al (2002). Cilengitide is a cyclic peptide that binds to αvβ3 and αvβ5. Abegrin™ is a monoclonal antibody directed against the αᵥβ₃ integrin and has been investigated in clinical studies in patients with advanced malignancies.

Taken together, the diverse integrin antagonists possess different intrinsic activities with respect to induction of apoptosis and/or anoikis (i.e. a form of programmed cell death which is induced by anchorage-dependent cells detaching from the surrounding extracellular matrix) which varies according to cell type. In addition, due to the close structural relationship (homology) between different integrin subunits and overlapping—and possibly redundant—physiological functions, it is difficult to predict the effects of different analogues of integrin inhibitors for every cell type. It is not yet clear whether endothelial cell apoptosis during in vivo angiogenesis results from the induction of death by pro-apoptotic factors, or by the inhibition of pro-survival factors, or both.

AV-398 was identified as potential αᵥβ₃ integrin inhibitor in vitro in U.S. patent application Ser. No. 11/559,857.

There is a continuing need for compositions and methods to treat diseases and disorders associated with increased angiogenesis of the eye and skin.

### SUMMARY OF THE INVENTION

One embodiment of the present invention is directed to composition and methods treating diseases or disorders associated with vascularization of the eye by controlling vascularization in a patient's eye. In general, the treatment methods for controlling vascularization of a patient's eye comprise topically and/or locally administering to a patient in need thereof an effective amount of a pharmaceutical composition comprising AV-398 and a suitable carrier.

Another embodiment of the present invention is directed to composition and methods treating diseases or disorders associated with vascularization of the skin by controlling vascularization in a patient's skin. In general, the treatment methods for controlling vascularization in a patient's skin comprise topically administering to a patient in need thereof an effective amount of a pharmaceutical composition comprising AV-398 and a suitable carrier.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a graph showing the comparative effects of AV-398 and echistatin on survival of human umbilical vein endothelial cells.

FIG. 2 is a graph showing the comparative effects of AV-398 and echistatin on survival of lymphoendothelial cells.

FIG. 3 is a graph showing the comparative effects of AV-398 and echistatin on survival of lung blood endothelial cells.

FIG. 4 shows AV-398-treated eyes and control-treated (vehicle alone) eyes 48 hrs after incision. Numbers #1 to #3 correspond to separate animals. AV-398 containing eye drops were applied in the right eye of each animal and control solutions into the left eye.

FIG. 5 shows AV-398 treated and vehicle control treated chicken embryos. Eggs were inspected daily by stereo-microscopy and pictures were taken (6× magnification). AV-398 treated embryos exhibited markedly decreased vascularization.

### DETAILED DESCRIPTION

One aspect of the present invention is directed to treatment methods and compositions for controlling vascularization in a patient's eye wherein the patient has a disease or disorder associated with vascularization in the eye or wherein said patient is at risk for developing a disease or disorder associated with vascularization. As used herein, the term “controlling vascularization” should be broadly interpreted to include inhibiting neovascularization in the eye, inhibiting angiogenesis in the eye, and eliminating, or diminishing the number or amount of existing pathological blood vessels. Preferably, when used to inhibit neovascularization or angiogenesis, the treatment methods of the present invention inhibit all or substantially all neovascularization and/or angiogenesis. However, the term “inhibiting” should be understood to encompass instances where there is less neovascularization or angiogenesis than in populations with the untreated disease or disorders.

In general, the treatment methods for controlling vascularization of a patient's eye comprise topically and/or locally administering to a patient in need thereof an effective amount of a pharmaceutical composition comprising AV-398 and a suitable carrier. The term “treatment” as used herein refers to both therapeutic treatment and prophylactic or preventative measures, wherein the objective is to prevent or slow down (lessen) the targeted pathologic condition or disorder. Those in need of treatment include those already with the disorder as well as those prone to have the disorder or those in whom the disorder is to be prevented.

Another aspect of the present invention is directed to methods and compositions for controlling vascularization in a patient's skin wherein the patient has a disease or disorder associated with vascularization in the skin or wherein said patient is at risk for developing a disease or disorder associated with vascularization in the skin. In general, the treatment methods for controlling vascularization in a patient's skin comprise topically administering to a patient in need thereof an effective amount of a pharmaceutical composition comprising AV-398 and a suitable carrier.

Although the present invention contemplates treatment methods involving only a single application of the pharmaceutical composition, the treatment methods of the present invention preferably include multiple applications of the pharmaceutical during the time the patient has the disease or disorder associated with vascularization or is at risk for such disease or disorder. Preferably, the multiple applications are spaced at intervals throughout the time the patient has need for said treatment. For instance, the pharmaceutical compositions may be applied two, three, four or more times per day during a course of treatment.

The pharmaceutical compositions of the present invention comprise AV-398 or a pharmaceutically acceptable salt thereof. AV-398 may be represented as having the following chemical structure:

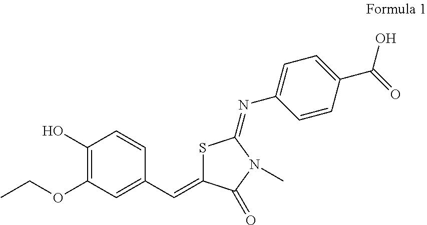 [SMILES: CCOc1cc(/C=C2\S/C(=N/c3ccc(C(=O)O)cc3)N(C)C2=O)ccc1O]

As used herein, the term “AV-398” should be interpreted to mean the compound of Formula 1 and pharmaceutically acceptable salts, hydrates, enantiomers, diastereomers, racemates or mixtures of stereoisomers thereof (note that in U.S. Provisional Application No. 61/549,617, the compound designated herein as AV-398 was designated as 0-398). The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

AV-398 may be formulated using either its acid or its mono-salt forms. Pharmaceutically acceptable salts may be obtained using standard procedures well known in the art. When used in its salt form, the salt may be any pharmaceutically acceptable suitable salt known to those of ordinary skill in the art so long as the AV-398 salt form is stable (i.e. does not degrade) and, when formulated as a solution, it is preferably sufficiently soluble in the pharmaceutical carrier to enable the delivery of an effective amount of AV-398 to the anatomical region to be treated. For example, equimolar concentrations of AV-398 and NaOH may be mixed in order to form a sodium salt of AV-398 at concentrations up to 50 mM. The Na-salt of AV-398 may then be directly dissolved in water, phosphate buffered saline or other physiological buffers, or in suitable pharmaceutical formulations. Suitable salts include mono-salts, such as potassium salts, sodium salts, and others.

An “effective amount” of the pharmaceutical composition comprising AV-398 is an amount sufficient to carry out the stated purpose of the treatment, i.e. therapeutic treatment or prophylactic or preventative measures relating to vascularization of the eye or skin. An “effective amount” may be determined empirically in relation to the stated purpose. As disclosed herein, AV-398 displays a 50% inhibitory concentration of about 0.2 to 0.5 μM in endothelial cell lines, demonstrating a high specificity against this type of cells. As such, the concentration of AV-398 in the pharmaceutical composition is preferably at least 0.2 to 0.5 μM. More preferably, when applied locally the anatomic region to be treated, the pharmaceutical composition should be sufficiently concentrated so that the concentration of AV-398 at the anatomical region to be treated is at least 0.2 to 0.5 μM. Further, when given as a course of multiple applications over a period of time, it is preferred that the concentration is greater than 0.2 to 0.5 μM during the entire course of treatment. Even more preferably, the pharmaceutical composition should be sufficiently concentrated to substantially control vascularization at the region to be treated throughout the course of treatment.

Sufficiently concentrated solutions are readily achievable. AV-398 is soluble in PBS 7.4 at 100 μg/ml, in Tris.Cl buffer at 1 mg/ml, and as a sodium salt at least up to 20 mg/ml. Moreover, as disclosed herein, the antiproliferative effects of AV-398 are generally specific to endothelial cell lines, and in vivo experiments in large animals, as described herein, indicate that multiple applications of AV-398 to the eye do not result in long term effects on non-endothelial cells or to other eye structures.

The pharmaceutical compositions of the present invention are preferably administered topically and/or locally. As used herein, topical administration refers to administration onto or into the eye, skin or nose. For purposes of this application, topical administration includes intradermal and intravitreal (or intraocular) injection. Further, for purpose of this application, local administration means to administer the pharmaceutical composition at or near the region to be treated. Systemic oral administration for the treatment of diseases of the eye or the skin is generally disfavoured due to the short plasma half-life and low oral absorption of AV-398. As described herein, terminal plasma half-life after intravenous administration of AV-398 was 0.5 h. There were no side-related effects on the hematological and serum chemistry parameters evaluated in rats treated with AV-398. The oral absorption of AV-398 when administered at 20 mg/kg dose as a suspension was low at an average of 8.4%. The pharmaceutical compositions of the present invention are generally formulations in which AV-398 is combined with a pharmaceutically acceptable carrier vehicle. Pharmaceutical compositions and formulations for topical administration can include ointments, lotions, creams, gels, drops, sprays, liquids and powders. Conventional pharmaceutical carriers, aqueous, powder or oily bases, thickeners and the like can be necessary or desirable.

Therapeutic formulations are generally prepared for storage by mixing the active ingredient, AV-398, having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are preferably nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, dextrins or cyclodextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for administration by injection may preferably be sterile. This may readily accomplished by filtration through sterile filtration membranes.

Generally, the concentration of AV-398 in a liquid composition, such as a solution, lotion or eyedrops, will be from about 0.1 μM to 1 mM, preferably from about 0.2 to 80 μM. The concentration in a semi-solid such as a gel or ointment will be about 0.1 μM to 1 mM, preferably about 0.2 to 80 μM. However, the amount of AV-398, or an active salt or derivative thereof, required for use in treatment will vary not only with the particular salt selected but also with the route of administration, the nature of the condition being treated and the age and condition of the patient and will be ultimately determined by the treating physician.

The treatment methods of the present invention may suitably be used to treat disorders associated with vascularization, including neovascularization, of the anterior segment of the eye. Thus, a further embodiment of the present invention includes methods and compositions for treating diseases or disorders associated with vascularization in the front third of the eye, including the cornea, iris, ciliary body and lens, and most preferably corneal vascularization. When used in connection with treatment for corneal vascularization, the pharmaceutical compositions are preferably eye drops administered topically. Treatment includes topical administration of an effective amount of the pharmaceutical composition on the outer surface of the affected eye and may optionally include treatment with other or additional therapeutics for treatment of the disease state. As shown herein, in vivo experiments demonstrate that pharmaceutical compositions of the present invention eliminate or prevent corneal neovascularization. Thus the treatment methods of the present invention may be used for treatment of vascularization in diseases causing corneal inflammation (e.g., bacterial and viral forms of keratitis), diseases interfering with the limbal barrier between normally avascular cornea and physiologically vascularized conjunctiva (e.g., chemical burns or inherited forms of limbal deficiency) and finally diseases presumably leading to corneal hypoxia (i.e., contact lenses with low oxygen penetration).

The treatment methods of the present invention may also suitably be used to treat disorders associated with vascularization of the posterior segment of the eye. Thus, a further embodiment of the present invention includes methods and compositions for treating diseases or disorders associated with vascularization in the back two-thirds of the eye, including the anterior hyaloid membrane and all of the optical structures behind it, including the retina, choroid, and optic nerve.

Specifically, the methods of the present invention may be used to treat disorders associated with the growth of blood vessels including, macular degeneration generally, including AMD, proliferative diabetic retinopathy (PDR), and retinopathy of prematurity (ROP). When used in connection with treatment of diseases of the posterior segment, treatment generally includes administration by injection of a liquid pharmaceutical composition at or near the affected portion of the eye. The injection preferably provides an effective amount of the pharmaceutical composition to the affected area and may optionally include treatment with other or additional therapeutics for treatment of the disease state.

The treatment methods of the present invention may also suitably be used to treat disorders associated with vascularization of the skin. For instance, the compositions of the present invention may be used to treat diseases such as rosacea, psoriasis and basal cell carcinoma, especially rosacea. Treatment generally includes administration by topical application of a cream, lotion, ointment or liquid at or near the affected portion of the skin. The composition preferably provides an effective amount of the pharmaceutical composition to the affected area and may optionally include treatment with other or additional therapeutics for treatment of the disease state. Other dermatological diseases associated with new blood vessel formation that may be treated using the compositions and treatment methods of the present invention include dermatitis (including atopic dermatitis and eczematous dermatitis), autoimmune skin diseases, acute and chronic urticaria, scleroderma, vasculitis, port-wine stains, blue rubber bleb syndrome, Osler-Weber-Rendu syndrome, Sturge-Weber syndrome Klippel-Trenaunay syndrome, non-melanoma skin cancer other than basal cell carcinoma, malignant melanoma, haemangiomas, angiosarcoma, pyogenic granuloma, viral warts, and keloid scars.

### Representative Synthesis of AV-398

### 1. Synthesis of 4-{[(methylamino)carbothioyl]amino}benzoic acid

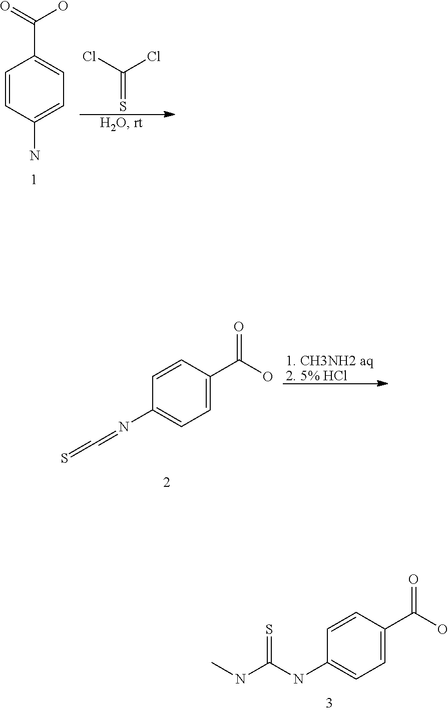

To a well-stirred suspension of 4-aminobenzoic acid (1) 13.7 g (100 mmol) in 300 ml of H₂O, 12.6 g (110 mmol) of thiophosgene is added dropwise (external cooling with ice-water bath to internal temperature between +10-+15° C.). Reaction mixture is then allowed to warm to room temperature (RT) and additionally stirred at RT overnight (approx. 18 h). To the resulting slurry of (2) with external cooling (internal temperature about 5° C.) a 30% water solution of methylamine (62 g, 600 mmol) is added dropwise. Stirring is then continued until reaction mixture reaches ambient temperature (approx. 3-4 hours). Then the solution of methilamine salt is transferred to a separation funnel and washed with 3×100 portions of EtOAc. Aqueous layer is then freed from traces of solvent on rotor desiccator (20 mmHg, 50° C. bath temperature) and cooled to 5° C. with ice-water bath). Keeping this temperature, the solution is acidified to pH 3 with excess of 5% HCl. White solid is filtered off, washed with cold water and dried in vacuo to constant weight. Yield of (3) is about 18.6 g (88%). Target product is white powder, mp>170° C. (decomposed).

According to NMR-¹H integral data, described work-up leads to product with less then 1-3 m % of impurity.

### 2. Synthesis of 4-[(3-methyl-4-oxo-1,3-thiazolan-2-yliden)amino]benzoic acid

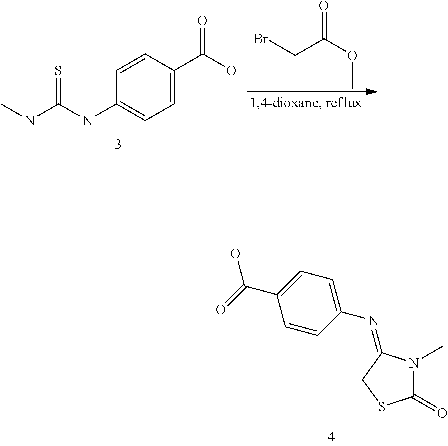

4-{[(Methylamino)carbothioyl]amino}benzoic acid (3) 10.5 g (50 mmol) and methyl-bromoacetate 9.2 g (60 mmol) in 150 ml of absolute dioxane are refluxed for 14 hours. After cooling of reaction mixture the solvent is removed under reduced pressure and yellow solid residue dissolved in 300 ml of 0.1N sodium hydroxide. The solution is extracted twice with EtOEt (2×100 ml) to remove all neutral organic components and then worked-up with activated charcoal. After acidification of alkaline solution to pH 5 with 5% HCl, (4) is filtered off and dried in vacuo. Yield of (4) is 8.6 g (69%). Product obtained is white powder, mp>130° C. (decomposed).

According to NMR-¹H integral data, described work-up leads to product with less then 1 m % of impurity.

### 3. Synthesis of 4-({5-[(E)-1-(3-ethoxy-4-hydroxyphenyl)methylidene]-3-methyl-4-oxo-1,3-thiazolan-2-yliden}amino)benzoic acid

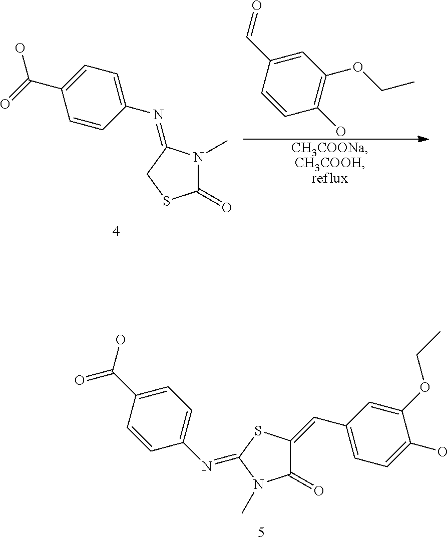

2.5 g (10 mmol) of (4), 1.99 g (12 mmol) of 3-ethoxy-4-hydroxybenzaldehyde and 1.64 g (20 mmol) of anhydrous sodium acetate are refluxed in acetic acid (50 ml) for 36 hours (until LCMS of reaction mixture shows no initial (4)). After cooling to room temperature the reaction mixture is poured in 200 ml of water, product (5) is filtered off, dried in vacuo and recrystallised from absolute EtOAc:hexane (5:1). Yield of yellow solid (mp. >180° C., decomp.) is 1.7 g (43%).

### EXAMPLES

The following examples are provided in order to demonstrate and further illustrate certain embodiments and aspects of the present invention and are not to be construed as limiting the scope thereof. Further, it is to be understood that this invention is not limited to the particular methodology, protocols, and reagents described, as these may vary.

### Example 1

### AV-398 is a Potent Specific Inhibitor of Endothelial Cell Growth

Cell Culture Methods:

Primary endothelial cell lines (HMVEC-dLyAd—human dermal lymphatic microvascular endothelial cells (EC), dermal blood microvascular EC, HMVEC-LB lung blood microvascular EC, HMVEC-C cardiac human microvascular endothelial cells and MVEC-d—dermal microvascular EC) are from Cambrex Bio Science (Cambrex Bio Science Walkersville Inc., Walkersville, Md., USA). Cell lines are cultured according to the manufacturer's recommendations. For all experiments, cells are used up to the sixth to eight passage, as suggested by the manufacturer, and harvested at 80% confluence.

For proliferation assays, cells are seeded in 24- to 96-well plates in medium at different concentrations according to the cell type. After 24 hours, the medium is removed and replaced with fresh medium containing increasing concentrations of the compounds. The plates are then incubated for additional 24 to 72 hours, depending on the type of the experiment. Cells are then trypsinized and collected for direct counting. Results are plotted and expressed as means (±SE) for each compound and for a given concentration.

Results:

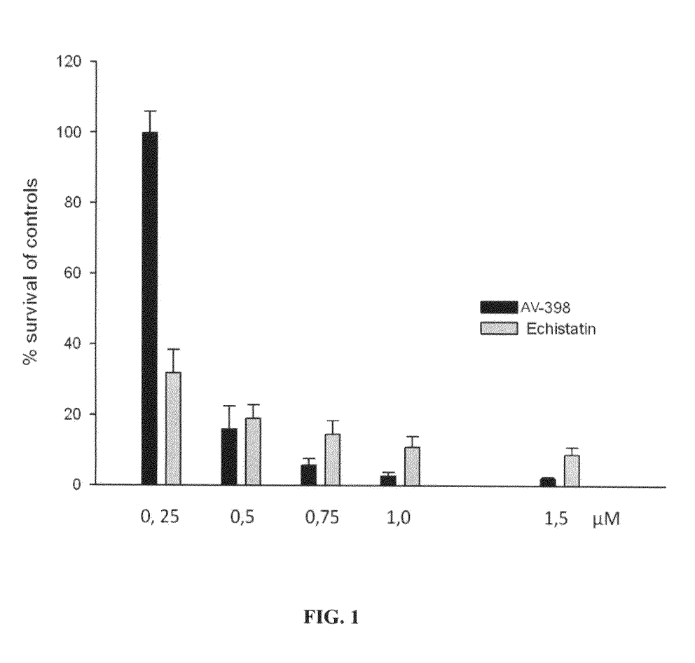

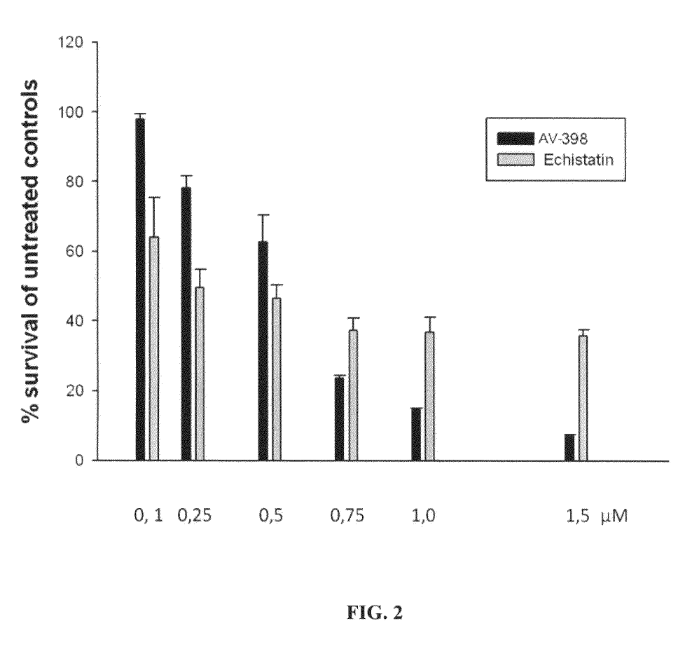

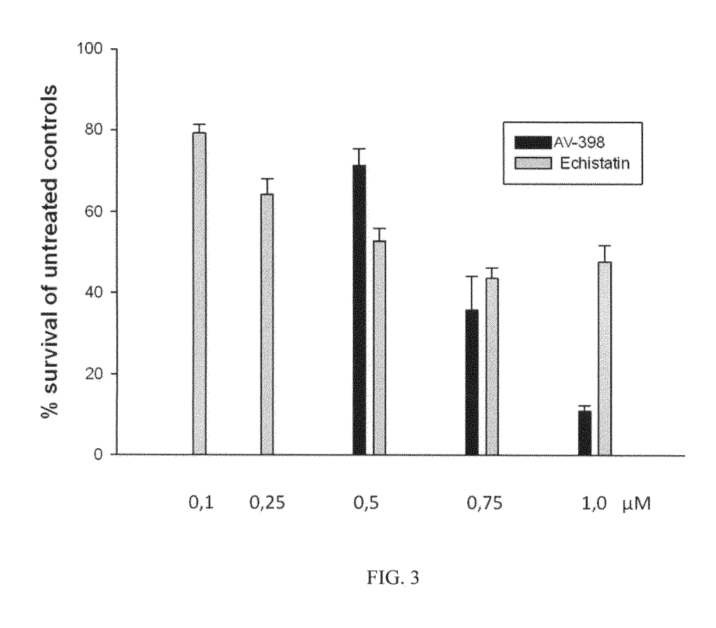

As shown in FIGS. 1-3, AV-398 displays a strong antiproliferative effect against endothelial cells of different origin (lung, lymphatics and umbilical) and is much less effective against most other cell types. Inhibition of lymphangiogenesis in addition to angiogenesis is of especial importance for certain diseases such as in corneal neovascularization (Birgit Regenfuss et al, 2008). Those figures also show that AV-398, on a molar basis, is more effective than the snake venom echistatin or cilengitide. Half-maximal growth inhibition for AV-398 was observed at 0.4 μM and with echistatin at 0.8 μM concentrations. These data suggest that both echistatin and AV-398 inhibit growth and adhesion of different endothelial cell lines, but with different efficacy. In contrast, and unexpectedly, several compounds which display a much higher affinity to the αvβ3 receptor, #3, #5 and #6 (see Table 3, original data published by Dayam et al, 2006)), delayed cellular adhesion, but affected the final number of endothelial cells adhered only weakly (up to 30%). In the presence of 0.5 to 1.0 μM concentrations of echistatin and AV-398, effects on cellular survival (morphological changes, detachment of cells) become apparent after approximately 12 to 16 h of addition of the compounds.

The inhibitory effect of AV-398 is highly and unexpectedly specific against endothelial cells as many integrin antagonists known form the literature do not display this selectivity against endothelial cells. For example, cytotoxic activity of AV-398 was determined in several cancer cell lines (SAOS-2, Hela, HT29, U87MG and SK-N-DZ cells) by assessing cell numbers after treating cell lines with varying concentration of up to 20 μM. Experiments were performed either on uncoated dishes, or on dishes coated with vitronectin or fibrinogen. In addition, tumor cell lines were treated in parallel with echistatin (Gan Z R et al (1998)). In general, no meaningful effects of AV-398 on cancer cell lines were observed, except limited activity against a single breast cancer cell line (MDA-MB-435). Similar data were recently published showing cytotoxic activity also only against MDA-MB-435 breast cancer cells (Dayam R et al, 2006).

In summary, AV-398 displays a 50% inhibitory concentration of 0.2 to 0.5 μM in endothelial cell lines, demonstrating a high specificity against this type of cells, which is unexpected, also in comparison to what is known from the literature about other integrin antagonists, such as cilengitide For example, Cilengitide reduced the colony-forming ability of endothelial cells with an IC50 of 6.7±1.2 μM as published by Tentori L et al (2008).

### Example 2

### Application of AV-398 Inhibits Ocular Angiogenesis in vivo

AV-398 inhibits ocular angiogenesis in vivo in a widely accepted large animal model. The cornea is an ideal model system for angiogenesis in general, and more specifically for angiogenesis of the eye. The corneal angiogenesis assay is one of the most widely used in vivo assay for the evaluation of factors influencing angiogenesis (Auerbach R, 2003, Thiele W, 2006). One reason that makes the cornea an ideal object for observing neovascularization is its normal lack of vascularity, maintained by different molecular mechanisms. New vessels can easily be identified by visual inspection alone and quantified with the help of computerized image analysis programs. The effect of an ophthalmological formulation of AV-398 after a conjunctival insult with subsequent angiogenesis in a large animal model was investigated in order to test whether the in vitro data, such as anti-proliferative effects on human endothelial cells, could be reproduced in vivo.

Animals and Methods:

Six white pigs weighing 10 to 15 kg were included. First, corneae and conjunctivae were anaesthesized by local application of lidocaine 4%. Then, an incision of 0.5 cm length through the conjunctival tunica sclerae to the substantia propria was placed in parallel to the dorsal corneal limbus at a distance of approximately 2 mm. The animals were treated as follows. The right eye of each animal was treated with a commercially available ocular eye drop solution (PROTAGENT® EYE DROPS 2%) containing 100 μg/ml of AV-398. The corresponding left eyes of each animal were control-treated with the same eye drop solution alone. Two droplets (˜100 μl) of AV-398 were applied in the right eye immediately after setting of the incision and two droplets of the ophthalmologic carrier formula as negative control in the left eye of the same animal. Animals received AV-398 as well as the eye drops 0.5 hrs post incision and the same treatment at 3 hrs post incision.

Results:

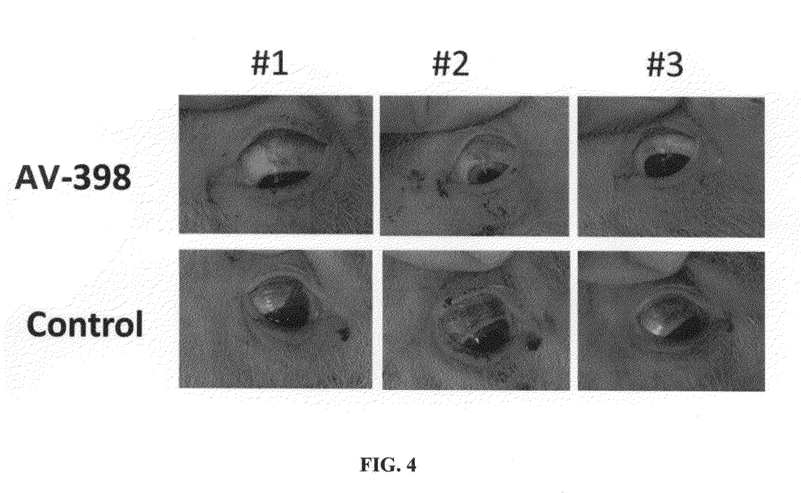

AV-398-treated pigs of group exhibited a marked diffuse bleeding surrounding the incision immediately after treatment. Episcleral vessels leading to the insult were thickened in both treated and control-treated eyes, but branched into newly formed capillaries in the AV-398-treated eyes to a lesser extent than those of the control eyes. All pigs showed a diminished neoangiogenesis on the AV-398-treated conjunctivae when compared to the control-treated eyes. Representative examples of three (out of total 6) AV-398 treated pigs are shown (FIG. 4) after 48 hrs. The inhibitory effect of AV-398 on angiogenesis could be clearly observed post treatment for several days. At day five, the control-treated eyes of pigs still displayed an increased neovascularization as compared to the AV-398-treated eyes. Three weeks post incision no local or systemic adverse side effects were visible in either control- or AV-398-treated animals.

### Example 3

### Topical Application of AV-398 Inhibits Neoangiogenesis in vivo

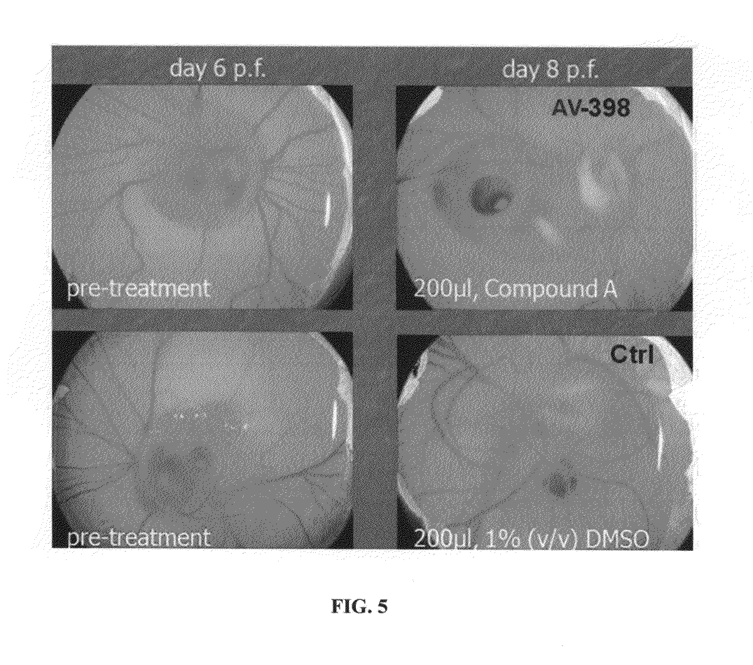

Local application of AV-398 inhibits in vivo angiogenesis as evidenced by the chicken egg chorioallantoic membrane model. The chorioallantoic membrane (CAM) of the developing chicken embryo serves as an alternative to the traditional mammalian in vivo models, and further corroborates the findings in pigs described above and has clear relevance for the treatment e.g. dermatological indications linked to neoangiogenesis. AV-398 was applied over 5 days. As shown in FIG. 5, a significant reduction of vascularization is seen in the AV-398 treated egg as compared to the control egg.

Experimental Procedure (CAM Assay):

Eggs were opened on day 6 post fertilization (p.f.). Substances (200 μl respectively) were applied on the CAM on day 6 p.f. and incubated until day 11 p.f. As a vehicle control, 1% DMSO in PBS (phosphate-buffered saline solution pH 7.4) without AV-398 was used. Eggs were inspected daily by stereo-microscopy and pictures were taken (6× magnification).

### Example 4

### Global Gene Expression Shows that AV-398 and Known Integrin Inhibitors have a Highly Significant Overlap of Genes and Proteins Regulated (Up or Down)

Global gene expression as well as proteome analysis of HUVECs treated with echistatin, and AV-398 clearly demonstrates a highly significant overlap of genes and proteins regulated by these compounds as is demonstrated in Table 1. For global gene expression analysis, endothelial cells were plated at low density and allowed to reach 60-80% confluence over 48 hr. Compounds of interest were then added together with fresh medium. At times of 8 and 16 h after start of the treatment, total RNA was isolated using the standard TRIzol procedure (Invitrogen, Carlsbad, Calif.). After a washing step with ice-cold PBS, TRIzol reagent was added directly to each well and gently agitated to aid in dissolution. RNA was extracted according to the manufacturer's protocol. For proteome analysis, total lysates were isolated after 14 hrs treatment with AV-398, echistatin or cilengitide. The table below shows proteins and/or genes which were regulated at least in two separate experiments by both AV-398 and echistatin. A select list of genes and protein names relevant for integrin dependent endothelial cell function is shown. These experiments demonstrate that AV-398 dependent mechanisms are regulated via integrin dependent pathways because most of the genes regulated are known from the literature to be involved in integrin-dependent signalling processes.

TABLE 1
Genes and Proteins Regulated by AV-398 and Echistatin

| | |
|---|---|
| Angiomotin-like Factor 2 | Galectin |
| Endothelin 1 | Ubiquitin |
| Selectin E | Cofilin |
| Hexokinase 2 | Cystein Rich Angiogenic Inducer 61 |
| Poliovirus Receptor | 7-Dehydrocholesterol Reductase |
| Sprouty Homolog 2 | A Kinase (PRKA) Anchor Protein 1 |
| Lipin 1 | PGAM (RNA and Protein) |
| CD7 | |

### Example 5

### There is a Stringent Dependence of Anti-Proliferative Effect on Chemical Structure

Minor changes in chemical structure of AV-398 have profound and unexpected effects on the anti-proliferative activity of the molecule. The stringent dependence of the antiangiogenic activity on chemical structure as determined by the anti-proliferative effects on human endothelial cell growth (HUVEC cell lines) is shown in Table 2, below. Very small changes in the structure lead to a pronounced loss of the desired activity. Compound number 3 is a representable example. For instance, while the IC50 for growth inhibition of AV-398 of HUVECs is below 0.3 μM, changes in the structure lead to an over 50-100-fold reduction of activity for most compounds tested. In Table 2, the group “R” is defined as follows:

TABLE 2

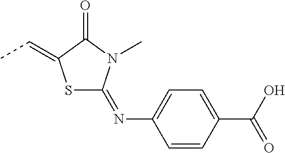

| Compound | | |
|---|---|---|
| Number | Structure | IC50 |
| #1 | 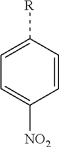 | >30 μM |
| #2 | 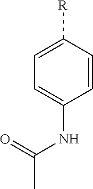 | >30 μM |
| #3 | 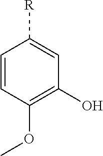 | >30 μM |
| #4 | 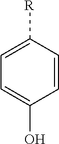 | >30 μM |
| #5 | 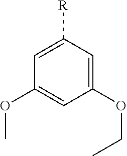 | >30 μM |
| #6 | 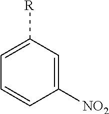 | >30 μM |
| #7 | 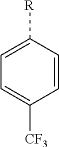 | >30 μM |
| #8 | 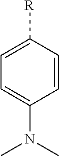 | ~20 μM |
| #9 | 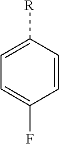 | >30 μM |
| #10 | 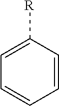 | >30 μM |
| #11 | 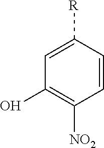 | >10 μM |
| #12 | 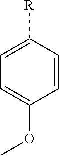 | >30 μM |

### Example 6

### Antiangiogenic Activity is not Predicted by Magnitude of Binding Affinity

Unexpectedly, the magnitude of the binding affinity to the cognate receptor does not predict the efficacy of the cell death inducing activity of AV-398 compared to other analogues. For instance, compound 6, which has a similar structure to AV-398 and an approximately 10,000-fold higher binding affinity to the αvβ3-receptor (0.03 nM for compound 6 compared to 240 nM for AV-398), unexpectedly displays far less cell death-inducing effects in endothelial cells. The binding affinities of similar or related compounds are shown below in Table 3.

TABLE 3
Comparison of Binding Affinity for various α_{υ}β₃ Antagonists (Dayam et al., 2006)

| | | α_{υ}β₃ binding affinity |
|---|---|---|
| Compd. | Structure | (nM) |
| 1 | 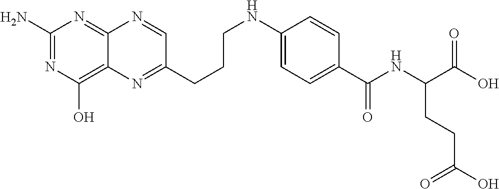 [SMILES: Nc1nc(O)c2nc(CCCNc3ccc(C(=O)NC(CCC(=O)O)C(=O)O)cc3)cnc2n1] | 52 |
| 2 (AV-398) | 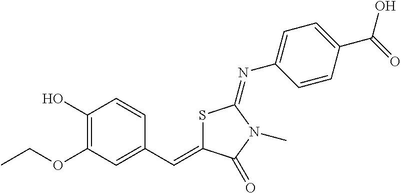 [SMILES: CCOc1cc(/C=C2\S/C(=N/c3ccc(C(=O)O)cc3)N(C)C2=O)ccc1O] | 240 |
| 3 | 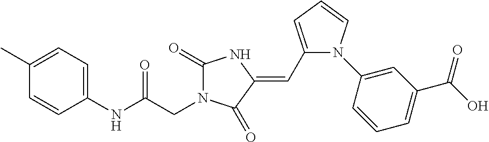 [SMILES: Cc1ccc(NC(=O)CN2C(=O)N/C(=C\c3cccn3-c3cccc(C(=O)O)c3)C2=O)cc1] | 18 |
| 4 | 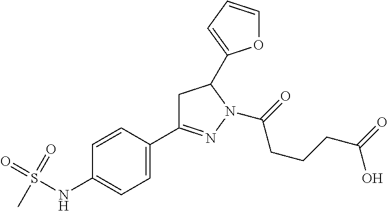 [SMILES: CS(=O)(=O)Nc1ccc(C2=NN(C(=O)CCCC(=O)O)C(c3ccco3)C2)cc1] | 605 |
| 5 | 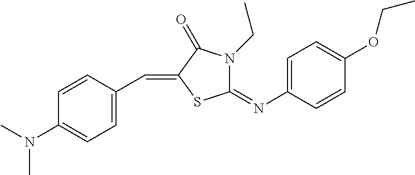 [SMILES: CCOc1ccc(/N=C2/S/C(=C\c3ccc(N(C)C)cc3)C(=O)N2CC)cc1] | 24 |
| 6 | 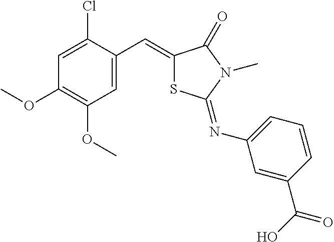 [SMILES: COc1cc(Cl)c(/C=C2\S/C(=N/c3cccc(C(=O)O)c3)N(C)C2=O)cc1OC] | 0.03 |

### Example 7

### Pharmacokinetic and Preliminary Toxicology Information

Preliminary pharmacokinetic and preliminary toxicology information was obtained following a single intravenous dose of 5 mg/kg and a single 20 mg/kg PO dose to male Sprague-Dawley rats for 5 separate test articles. AV-398 was dosed both via the IV and PO routes. Blood was collected from the animals in Groups 1 through 10 for pharmacokinetic analysis. Approximately 0.25 mL of blood was collected in potassium EDTA tubes via the jugular cannulae for all time points. For all IV treated animals, blood was collected at 9 time points (0.083, 0.25, 0.5, 1, 2, 4, 6, 8 and 12 hour post-dose) via the jugular cannulae. For all animals treated by oral gavage, blood was collected at 7 time points (0.5, 1, 2, 4, 6, 8 and 12 hour post-dose) via the jugular cannulae. At the final time point additional blood volume was collected in a separate EDTA tube from each animal for clinical pathology evaluation (including serum chemistry and hematology). Predose samples were collected from the extra animals which were not assigned to any of the dose groups. The plasma samples were analyzed by LC-MS/MS to determine the plasma concentrations of drug candidates. Pharmacokinetic analysis of the plasma concentration data was conducted using non-compartmental analysis with WinNonlin Version 4.1. After intravenous administration of AV-398 at 5 mg/kg, peak plasma concentrations were reached at 0.083 hr post-dose with an average concentration of 1736.56 ng/mL. Terminal plasma half-life was 0.5 hr, while the average AUC (0-∞) was 690.48 hr*ng/mL. After oral administration of AV-398 as a suspension at 20 mg/kg, peak plasma concentrations were reached at between 2 and 4 hr post-dose, with a mean concentration of 54.07 ng/mL. The average AUC (0-12 hr) was 233.61 hr*ng/mL. There were no side-related effects on the hematological and serum chemistry parameters evaluated in rats treated with AV-398. AV-398 when administered IV to rats at 5 mg/kg had a terminal plasma half-life of 0.18 to 0.68 hours. The oral absorption of AV-398 when administered at 20 mg/kg dose as a suspension was low at an average of 8.4%.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Those skilled in the art will recognize, or be able to ascertain using no more than routine experimentation, many equivalents to the specific embodiments of the method and compositions described herein. Such equivalents are intended to be encompassed by the following claims.

### REFERENCES

The following references are cited herein. The entire disclosure of each reference is relied upon and incorporated by reference herein.
Aplin A E, Howe A, Alahari S K, et al. Signal transduction and signal modulation by cell adhesion receptors: the role of integrins, cadherins, immunoglobulin-cell adhesion molecules, and selectins. Pharmacol Rev 1998 50:197-63.
Auerbach R, Lewis R, Shinners B, Kubai L, Akhtar N. Angiogenesis assays: A critical overview. Clin Chem 2003; 49:32-40.
Cai W, Wu Y, Chen K, et al. In-vitro and In-vivo Characterization of 64Cu-Labeled Abegrin™, a Humanized Monoclonal Antibody against Integrin αvβ3. Cancer Res 2006 66:9673-81.
Cheresh D A, Harper J R. Arg-Gly-Asp recognition by a cell adhesion receptor requires its 130-kDa alpha subunit. J Biol Chem 1987 262:1434-37.
Cheresh D A. Human endothelial cells synthesize and express an Arg-Gly-Asp-directed adhesion receptor involved in attachment to fibrinogen and von Willebrand factor. Proc Natl Acad Sci U S A 1987 84:6471-75.
Dayam R, Aiello F, Deng J, et al. Discovery of small molecule integrin αvβ3 antagonists as novel anticancer agents. J Med Chem 2006 49: 4526-34.
Eskens F A, Dumez H, Hoekstra R, et al. Phase I and pharmacokinetic study of continuous twice weekly intravenous administration of Cilengitide (EMD 121974), a novel inhibitor of the integrins integrins αvβ3 and αvβ5 in patients with advanced solid tumours. Eur J Cancer 2003 39:917-26.
Gan Z R, Gould R J, Jacobs J W, Friedman P A, Polokoff M A. Echistatin. A potent platelet aggregation inhibitor from the venom of the viper, Echis carinatus. J Biol Chem 1988 263:9827-32.
Hynes R O. Changing Partners. Science 2003 300:755-56.
Nabors L B, Mikkelsen T, Rosenfeld S, et al. Phase I and correlative biology study of cilengitide in patients with recurrent malignant glioma. J Clin Oncol 2007 25:651-57.
Birgit Regenfuss, Felix Bock, Anand Parthasarathy, and Claus Cursiefen. LYMPHATIC RESEARCH AND BIOLOGY Volume 6, Number 3-4, 2008.
Ruoslahti E. RGD and other recognition sequences for integrins. Annu Rev Cell Dev Biol 1996 12:697-15.
Schwartz M A, Shattil S J. Signaling networks linking integrins and rho family GTPases. Trends Biochem Sci 2000 25:388-91.
Smith J W. Cilengitide Merck. Curr Opin Investig Drugs 2003 4:741-45. Review.
Stromblad S, Becker J C, Yebra M, et al. Suppression of p53 activity and p21WAF1/CIP1 expression by vascular cell integrin aVh3 during angiogenesis. J Clin Invest 1996 98:426-33.
Taga T, Suzuki A, Gonzalez-Gomez I, et al. αv-integrin antagonist EMD 121974 induces apoptosis in brain tumor cells growing on vitronectin and tenascin. Int J Cancer 2002 98:690-97.
Tentori L, Dorio A S, Muzi A, et al. The integrin antagonist cilengitide increases the antitumor activity of temozolomide against malignant melanoma. Oncol Rep. 2008 19:1039-43.
Thiele W, Sleeman J P. Tumor-induced lymphangiogenesis: a target for cancer therapy? J Biotechnol 2006 24:224-41. Review.
Zedith J E, Jr., Fazeli B, Schwartz M A. The extracellular matrix as a cell survival factor. Mol Biol Cell 1993 4:953-61.

What is claimed is:

1. A method for controlling vascularization in a patient's eye or skin comprising administering to the patient's eye or skin a pharmaceutical composition having 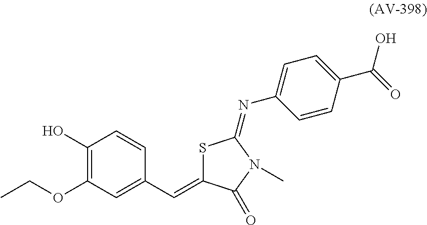 [SMILES: CCOc1cc(/C=C2\S/C(=N/c3ccc(C(=O)O)cc3)N(C)C2=O)ccc1O]
or a pharmaceutically acceptable salt, hydrate, enantiomer, diastereomer, racemate or mixtures of stereoisomers thereof;
wherein the patient has a disease or disorder associated with vascularization in the eye or skin or wherein said patient is at risk for developing a disease or disorder associated with vascularization of the eye or skin.

2. The method of claim 1, wherein the pharmaceutical composition controls vascularization by inhibiting neovascularization in the eye.

3. The method of claim 1, wherein the pharmaceutical composition controls vascularization by eliminating existing blood vessels.

4. A method for treating a disease or disorder associated with vascularization in a patient's eye comprising:
topically and/or locally administering to the patient's eye an effective amount of a pharmaceutical composition comprising AV-398, or a pharmaceutically acceptable salt thereof, and a suitable carrier.

5. The method of claim 4, wherein the pharmaceutical composition is a liquid and the carrier comprises water.

6. The method of claim 4, wherein the concentration of AV-398 in the pharmaceutical composition is in the range of about 0.1 μM to 1 mM.

7. The method of claim 6, wherein the pharmaceutical composition is administered by placing the pharmaceutical composition on the surface of the patient's eye.

8. The method of claim 7, wherein the pharmaceutical composition is an eyedrop.

9. The method of claim 6, wherein the pharmaceutical is administered by intraocular or intravitreal injection.

10. The method of claim 4, wherein the concentration of AV-398, or the pharmaceutically acceptable salt, at or near a region of the eye to be treated is at least 0.2 to 0.5 μM.

11. A method for treating a disease or disorder associated with vascularization in a patient's skin comprising:
topically and/or locally administering to the patient's skin an effective amount of a pharmaceutical composition comprising AV-398, or a pharmaceutically acceptable salt thereof, and a suitable carrier.

12. The method of claim 11, wherein the concentration of AV-398, or the pharmaceutically acceptable salt, in the pharmaceutical composition is in the range of about 0.1 μM to 1 mM.

13. The method of claim 12, wherein the pharmaceutical composition is administered by placing the pharmaceutical composition on the surface of the patient's skin.

14. The method of claim 13, wherein the pharmaceutical composition is in the form of a lotion, cream or ointment.

15. The method of claim 4, wherein the concentration of AV-398, or the pharmaceutically acceptable salt, at or near a region of the skin to be treated is at least 0.2 μM.

16. A pharmaceutical composition for controlling vascularization in a patient's eye or skin comprising: 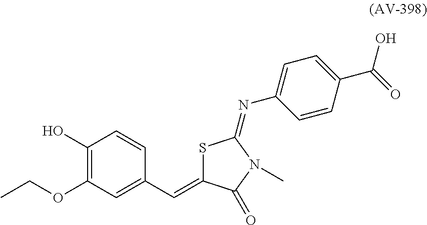 [SMILES: CCOc1cc(/C=C2\S/C(=N/c3ccc(C(=O)O)cc3)N(C)C2=O)ccc1O]
or a pharmaceutically acceptable salt thereof; and
a carrier.

17. The composition of claim 16, wherein the concentration of AV-398, or the pharmaceutically acceptable salt, in the pharmaceutical composition is in the range of about 0.1 μM to 1 mM.

18. The composition of claim 17, wherein the pharmaceutical composition is a liquid and the carrier comprises water.

19. The composition of claim 18, wherein the pharmaceutical composition is an eyedrop.

20. The composition of claim 16, wherein the pharmaceutical composition is in the form of a lotion, cream or ointment.
